(19) United States
(12) Patent Application Publication
(10) Pub. No.: US 2021/0128685 A1
(43) Pub. Date: May 6, 2021

(54) PEDF-DERIVED PEPTIDES FOR PROMOTING MEIBOMIAN GLAND REGENERATION AND USES THEREOF

(71) Applicant: BRIM Biotechnology, Inc., Taipei (TW)

(72) Inventors: Nai-Wen Fan, Taipei (TW); Tsung-Chuan Ho, Taipei (TW); Yeou-Ping Tsao, Taipei (TW); Frank Wen-Chi Lee, Bedford, MA (US)

(73) Assignee: BRIM Biotechnology, Inc., Taipei (TW)

(21) Appl. No.: 17/053,060

(22) PCT Filed: May 4, 2019
(86) PCT No.: PCT/US19/30767

§ 371 Date: Nov. 4, 2020

Related U.S. Application Data
(60) Provisional application No. 62/667,415, filed on May 4, 2018.

(51) Int. Cl. A61K 38/18; A61P 27/02

(52) U.S. Cl. CPC A61K 38/18; A61P 27/02

(57) ABSTRACT

A pharmaceutical composition or method for promoting meibomian gland regeneration or for treating/preventing dry eye syndrome includes administering to a subject in need thereof a pharmaceutical composition comprising a PEDF-derived short peptide (PDSP) or a variant of the PDSP, wherein the PDSP comprises residues 93-106 of human pigmented epithelium-derived factor (PEDF).

### FIELD OF THE INVENTION

This invention relates to PELF-derived peptides and their uses in Meibomian gland regeneration or in the treatment of dry eyes.

### BACKGROUND OF THE INVENTION

Meibomian gland dysfunction (MGD) is characterized by decreased quantitative and/or qualitative changes of meibomian gland secretions, instability of tear film lipid layer, and symptoms of eye irritation.¹⁻³ Since MGD accounts for as much as two-thirds of all case with dry eye disease (DED), it is considered a growing public issue, especially in older population.^{1,2} However, currently clinical MGD treatment modalities, including topical medication, Meibomian gland (MG) expression, Lipiflow, and intense pulsed light (IPL) treatment, are mostly palliative, as they often aim primarily at symptomatic relief of DED, preventing further MG atrophy, and not directly at remediating the underlying pathogenesis of MGD.^{1,2,4}

There are three forms of MGD: hypersecretory MGD, hyposecretory MGD, and obstructive MGD.⁵ Obstructive MGD is considered the most common and thought to be involved in hyperkeratinization of the duct orifice, causing ductal obstruction and further acinar atrophy.^{3,5} However, the findings of anterior displacement of mucocutaneous junction in patients with MGD and non-keratinized ductal epithelial cells at the orifice of murine MG do not support the conventional theory of hyperkeratinization as a primary mechanism for MGD.^{5,6} For age-related MGD, gland atrophy with decreased cell proliferation were observed in both human and murine Meibomian glands.^{7,8} Acinar tissue atrophy may be the primary etiology that results in an imbalance between lipid and ductal cells, or alteration of lipids/protein ratio contributing to the plugs at the orifice.^{5,9}

While meibomian gland dysfunction (MGD) affects many patients, current treatments are mostly palliative. Therefore, there is a need for more effective treatments for MGD.

### SUMMARY OF INVENTION

Embodiments of the invention relate to methods for promoting Meibomian gland regeneration and for treating dry eyes using short peptides derived from pigment epithelium-derived factors (PEDF).

One aspect of the invention relates to methods for promoting Meibomian gland regeneration. A method in accordance with one embodiment of the invention includes administering to a subject in need thereof a pharmaceutical composition comprising a PEDF-derived short peptide (PDSP) or a variant of the PDSP, wherein the PDSP comprises residues 93-106 of human pigmented epithelium-derived factor (PEDF).

One aspect of the invention relates to methods for treating dry eye syndromes. A method in accordance with one embodiment of the invention comprises administering to a subject in need thereof a pharmaceutical composition comprising a PEDF-derived short peptide (PDSP) or a variant of the PDSP, wherein the PDSP comprises residues 93-106 of human pigmented epithelium-derived factor (PEDF).

Other aspect of the invention will become apparent with the following detailed description and the attached claims.

### BRIEF DESCRIPTION OF THE DRAWINGS

FIG. 1 shows MG atrophy and tear film instability in aged mice. Panel (A) shows upper and lower eyelids harvested from young and old mice stained with Oil-Red-O (ORO). Meibum (red) was visible in the main duct (arrows) and acini (arrow heads). Representative images are from 7 eyelids of old mice and 6 eyelids of young mice. Panel (B) shows the grading of Meibomian gland atrophy as calculated and converted into the MG scores based on the percentages of atrophy areas. Panel (C) shows cryosections of upper eyelids from old and young mice stained with ORO. Panel (D) shows tear film break-up time as evaluated from 16 eyes of 8 young mice and 12 eyes of 6 old mice. Data are reported as mean±SE. *P<0.05 versus young mice. **P<0.001 versus young mice.

FIG. 2 shows PEDF expression in upper eyelids of young and old mice. Representative PEDF-stained cross sections showed the acini of young mice (A) and old mice (B). Immunostaining with second antibody alone was served as a negative control (C). Boxes in the low magnification images (upper panel) indicate the locations of the higher magnification images (lower panels), which show that PEDF is expressed mainly in the nucleus of progenitor cells (black arrows; (D) and (E)). PEDF expression was visualized in whole acini and stronger in cytoplasm at acinar base. (F) The PEDF histopathological scores were calculated based on the staining intensity, intensity of cytoplasm at acinar base, and the percentage of PEDF-positive cell nucleus. Three randomized images were captured from each eyelid for analysis. Representative images are from 6 eyelids of 6 different mice in each group (original magnification: ×400). *P<0.05 versus young group. Scale bar, 50 μm.

FIG. 3 shows that the 29-mer promotes acinar progenitor cell proliferation in aged mice. To detect DNA synthesis, BrdU was intraperitoneally injected immediately after 29-mer treatment and eyelids were harvested at 24 hours. Under normal conditions (without any treatment), old mice (A) exhibited fewer BrdU-positive cells than young mice (D). 29-mer increased BrdU-positive cells in acinar base of old (C) and young mice (F), while DMSO had no effect (B and E). Red broken-line circles indicated the central ducts. Black arrows indicated BrdU-positive progenitor cells. (G) The PEDF treatment effects were calculated based on the BrdU-positive cells in acinar base. Three randomized images were captured from each eyelid for analysis. Representative images are from 6 eyelids of 6 different mice in each group (original magnification: ×400). Representative images are from 3 eyelids of 3 different mice in each group (original magnification: ×400). #P<0.05 versus young mice normal condition, *P<0.05 versus old mice treated with DMSO or 18-mer. **P<0.05 versus young mice normal condition, or young mice treated with vehicle DMSO. Scale bar, 50 μm.

FIG. 4 shows cell proliferation in upper eyelids 5 days after treatments. After single subconjunctival injection, BrdU was intraperitoneally injected at day 0 and day 3. Then, eyelids were harvested at day 5. (A) and (C) show the control with DMSO treatments for old and young mice, respectively. (C) and (D) show PDSP treatments in old and young mice, respectively. The BrdU-positive cell were mainly the acinar progenitor cells (black arrows) and a few meibocytes were positive for BrdU staining (D, red arrow). (E) The PEDF treatment effects were calculated based on the BrdU-positive cells in acinar base. Three randomized images were captured from each eyelid for analysis. Representative images are from 3 eyelids of 3 different mice in each group (original magnification: ×400). *P<0.001 versus old mice treated with DMSO. Scale bar, 50 μm.

FIG. 5 shows immunohistochemistry analysis of stem cell marker p63 expression in upper eyelids 5 day after single treatment. (A) and (B) show baseline p63 expressions in old and young mice, respectively. (C) and (D) show DMSO and PDSP treatments in old mice, respectively. Red broken-line circles indicated the central ducts. (E) The number of p63-positive cells per acinus was evaluated. Representative images are from 3 eyelids of 3 different mice in each group (original magnification: ×400). Three randomized images were captured from each eyelid for analysis. *P<0.001 versus young mice, **P<0.001 versus old mice treated with DMSO. Scale bar, 50 μm.

FIG. 6 shows that 29-mer increases tear film stability of aging mice. Levels of tear break-up time (TBUT) (A) and tear volume secretion (B) are shown for weeks 1, 2, 3, 4, and 8 after the 29-mer injection. Values are expressed as mean±SE. Data are from 6 eyelids of 6 different mice in each group. Three measurements of tear film break-up time from one eyelid were recorded. *P<0.05 versus vehicle group at same time point.

FIG. 7 shows that the 29-mer increases acinar size of aged mice. (A) Left, whole-mount ORO staining for upper eyelids. Right, MG area was analyzed using area calculation tool in Adobe Photoshop 7.0. (B) shows the histogram of MG size of upper eyelids measured by Photoshop was presented in Pixels. Cryosections of upper eyelids were stained with ORO. Histogram of acinar size measured by Adobe Photoshop 7.0 was presented in Pixels. Representative images of whole-mount are from 7 eyelids of 7 different mice in each group. Representative images of cryosections are from 3 eyelids of 3 different mice in each group. *P<0.05 versus vehicle group.

### DETAILED DESCRIPTION

Embodiments of the invention relates methods for promoting meibomian gland regenerations using PEDF-derived short peptides (PDSP). Meibomian glands are a holocrine type exocrine glands. Meibomian glands are located at the rim of the eyelids inside the tarsal plate and are responsible for the supply of meibum, an oily substance that prevents evaporation of the tear films on the eyes. Meibomian gland dysfunction (MGD) is the most common cause of dry eye syndrome (or dry eye disease). MGD may lead to eyelid inflammation, called blepharitis, especially along the rims.

In normal MGs homeostasis, meibocytes within MG acini are continuously differentiated from the stem cells in the basal cell layer in the periphery of the acinus.⁴ Here, for the first time, we found that PEDF protein expression mainly in the nucleus of acinar basal cells (progenitor cells) and the cytoplasm at the acinar base. With aging change, the expression of PEDF protein reduced significantly.

Human Pigment Epithelium-derived Factor (PEDF) is a secreted glycoprotein containing 418 amino acids, with a molecular weight of about 50 kDa. PEDF is a multifunctional protein, which was first identified and isolated from the conditioned medium of culture human fetal retinal pigment epithelial cells.¹⁰⁻¹² The PEDF was broadly expressed in liver, adipose tissue, eye, heart, pancreas and plays fundamental roles in organogenesis and homeostatic maintenance of adult tissue.¹²⁻¹⁴

The different motifs of PEDF exert different biological activities. For example, a 44-mer motif (amino acid positions Val⁷⁸-Thr¹²¹) determines the neurotrophic and mitogenic activity of PEDF.^{12,15} On the other hand, a 34-mer fragment (residues 44-77 of PEDF) has been identified to have anti-angiogenic activity. We found that the 44-mer (Val⁷⁸-Thr¹²¹) could induce stem cells proliferation and regeneration in the limbus of rabbit.¹⁶⁻¹⁸ Further, a shorter peptide 29-mer (residues Ser⁹³-Thr¹²¹) was found to induce proliferation of myogenic stem cells and C2C12 myoblasts.¹⁵ The present invention was based on the finding that PEDF protein expression in MG acini reduces with aging.

Inventors of the present invention unexpectedly found that certain PEDF-derived short peptides (PDSPs) can increase proliferation of acinar progenitor cells as well as acinar size and tear-film stability in vivo. These PDSPs can promote meibomian gland regenerations and can be used to treat or prevent dry eye diseases.

The PDSPs of the invention are based on the peptide region corresponding to human PEDF residues 93-121 (⁹³SLGAEQRTESIIHRALYYDLISSPDIHGT¹²¹; SEQ ID NO:1). Based on this 29-mer, inventors identified that serine-93, alanine-96, glutamine-98, isoleucine-103, isoleucine-104, and arginine-106 are critical for the activities, as evidenced by significant loss of activities when these residues were individually replaced with alanine (or glycine for Alanine-96). In contrast, alanine (or glycine) replacements of other residues in the 29-mer did not appreciably change the activities, suggesting PDSP variants having amino acid substitutions (particularly, homologous amino acid substitutions) at these other residues (i.e., residues 94, 95, 97, 99-102, 105, and 107-121) can also be used to prevent and/or treat osteoarthritis, or to induce chondrogenesis.

These results indicate that the core peptide containing the antinociceptive effects is in the region comprising residues 93-106 (⁹³SLGAEQRTESIIHR¹⁰⁶; SEQ ID NO:2). Thus, the shortest PDSP peptide having the antinociceptive activity may be a 14-mer. One skilled in the art would appreciate that addition of additional amino acids to this core peptide, at the C and/or N terminus, should not affect this activity. That is, a PDSP of the invention may be any peptide comprising residues 93-106 of human PEDF. Therefore, a PDSP peptide for the invention may be a 14-mer, 15-mer, 16-mer, and so on, including the 29-mer used in the experiments.

Furthermore, as noted above, substitutions within these short peptides can retain the activities, as long as the critical residues (serine-93, alanine-96, glutamine-98, isoleucine-103, isoleucine-104, and arginine-106) are preserved. In addition, the mouse variants (which have two substitutions: histidine-98 and valine-103, as compared with the human sequence) are also active. The corresponding mouse sequences are: mo-29mer (SLGAEHRTESVIHRALYYDLITNPDIHST, SEQ ID NO: 3) and mo-14mer (SLGAEHRTESVIHR, SEQID NO: 4). Thus, a generic sequence for an active core is (⁹³S-X-X-A-X-Q/H-X-X-X-X-I/V-I-X-R¹⁰⁶, wherein X represents any amino-acid residue; SEQ ID NO: 5). A few examples of PDSP sequence that may be used with embodiments of the invention are shown in the following Table (the positions numberings are based on the positions in the 14mers). These examples are not meant to be limiting.

| | SEQ |
|---|---|
| | ID |
| Peptide Sequences | NO |
| ¹S-²X-³X-⁴A-⁵X-⁶Q/H-⁷X-⁸X-⁹X-¹⁰X-¹¹I/V-¹²I-¹³X-¹⁴R | 5 |
| ¹S-²L-³X-⁴A-⁵X-⁶Q/H-⁷X-⁸X-⁹X-¹⁰X-¹¹I/V-¹²I-¹³X-¹⁴R | 6 |
| ¹S-²A-³X-⁴A-⁵X-⁶Q/H-⁷X-⁸X-⁹X-¹⁰X-¹¹I/V-¹²I-¹³X-¹⁴R | 7 |
| ¹S-²X-³G-⁴A-⁵X-⁶Q/H-⁷X-⁸X-⁹X-¹⁰X-¹¹I/V-¹²I-¹³X-¹⁴R | 8 |
| ¹S-²X-³A-⁴A-⁵X-⁶Q/H-⁷X-⁸X-⁹X-¹⁰X-¹¹I/V-¹²I-¹³X-¹⁴R | 9 |
| ¹S-²X-³X-⁴A-⁵E-⁶Q/H-⁷X-⁸X-⁹X-¹⁰X-¹¹I/V-¹²I-¹³X-¹⁴R | 10 |
| ¹S-²X-³X-⁴A-⁵A-⁶Q/H-⁷X-⁸X-⁹X-¹⁰X-¹¹I/V-¹²I-¹³X-¹⁴R | 11 |
| ¹S-²X-³X-⁴A-⁵X-⁶Q/H-⁷R-⁸X-⁹X-¹⁰X-¹¹I/I-¹²I-¹³X-¹⁴R | 12 |
| ¹S-²L-³X-⁴A-⁵X-⁶Q/H-⁷A-⁸X-⁹X-¹⁰X-¹¹I/V-¹²I-¹³X-¹⁴R | 13 |
| ¹S-²A-³X-⁴A-⁵X-⁶Q/H-⁷X-⁸T-⁹X-¹⁰X-¹¹I/V-¹²I-¹³X-¹⁴R | 14 |
| ¹S-²X-³G-⁴A-⁵X-⁶Q/H-⁷X-⁸A-⁹E-X¹⁰_¹¹I/V-¹²I-¹³X-¹⁴R | 15 |
| ¹S-²X-³A-⁴A-⁵X-⁶Q/H-⁷X-⁸X-⁹A-X¹⁰-¹¹I/V-¹²I-¹³X-¹⁴R | 16 |
| ¹S-²X-³X-⁴A-⁵E-⁶Q/H-⁷X-⁸X-⁹X-¹⁰S-¹¹I/V-¹²I-¹³X-¹⁴R | 17 |
| ¹S-²X-³X-⁴A-⁵A-⁶Q/H-⁷X-⁸X-⁹X-¹⁰A-¹¹I/V-¹²I-¹³X-¹⁴R | 18 |
| ¹S-²X-³X-⁴A-⁵X-⁶Q/H-⁷X-⁸X-⁹X-¹⁰X-¹¹I/V-¹²I-¹³H-¹⁴R | 19 |
| ¹S-²X-³X-⁴A-⁵X-⁶Q/H-⁷X-⁸X-⁹X-¹⁰X-¹¹I/V-¹²I-¹³A-¹⁴R | 20 |
| ¹S-²L-³G-⁴A-⁵X-⁶Q/H-⁷X-⁸X-⁹X-¹⁰X-¹¹I/V-¹²I-¹³X-¹⁴R | 21 |
| ¹S-²L-³G-⁴A-⁵E-⁶Q/H-⁷X-⁸X-⁹X-¹⁰X-¹¹I/V-¹²I-¹³X-¹⁴R | 22 |
| ¹S-²L-³G-⁴A-⁵A-⁶Q/H-⁷X-⁸X-⁹X-¹⁰X-¹¹I/V-¹²I-¹³X-¹⁴R | 23 |
| ¹S-²L-³G-⁴A-⁵X-⁶Q/H-⁷R-⁸X-⁹X-¹⁰X-¹¹I/V-¹²I-¹³X-¹⁴R | 24 |
| ¹S-²L-³G-⁴A-⁵X-⁶Q/H-⁷A-⁸X-⁹X-¹⁰X-¹¹I/V-¹²I-¹³X-¹⁴R | 25 |
| ¹S-²L-³G-⁴A-⁵X-⁶Q/H-⁷X-⁸T-⁹X-¹⁰X-¹¹I/V-¹²I-¹³X-¹⁴R | 26 |
| ¹S-²L-³G-⁴A-⁵X-⁶Q/H-⁷X-⁸A-⁹E-¹⁰X-¹¹I/V-¹²I-¹³X-¹⁴R | 27 |
| ¹S-²L-³G-⁴A-⁵X-⁶Q/H-⁷X-⁸X-⁹A-¹⁰X-¹¹I/V-¹²I-¹³X-¹⁴R | 28 |
| ¹S-²L-³G-⁴A-⁵X-⁶Q/H-⁷X-⁸X-⁹X-¹⁰S-¹¹I/V-¹²I-¹³X-¹⁴R | 29 |
| ¹S-²L-³G-⁴A-⁵X-⁶Q/H-⁷X-⁸X-⁹X-¹⁰A-¹¹I/V-¹²I-¹³X-¹⁴R | 30 |
| ¹S-²L-³G-⁴A-⁵X-⁶Q/H-⁷X-⁸X-⁹X-¹⁰S-¹¹I/V-¹²I-¹³H-¹⁴R | 31 |
| ¹S-²L-³G-⁴A-⁵X-⁶Q/H-⁷X-⁸X-⁹X-¹⁰A-¹¹I/V-¹²I-¹³A-¹⁴R | 32 |
| ¹S-²L-³G-⁴A-⁵E-⁶Q/H-⁷X-⁸X-⁹X-¹⁰X-¹¹I/V-¹²I-¹³X-¹⁴R | 33 |
| ¹S-²L-³G-⁴A-⁵E-⁶Q/H-⁷R-⁸X-⁹X-¹⁰X-¹¹I/V-¹²I-¹³X-¹⁴R | 34 |
| ¹S-²L-³G-⁴A-⁵E-⁶Q/H-⁷A-⁸X-⁹X-¹⁰X-¹¹I/V-¹²I-¹³X-¹⁴R | 35 |
| ¹S-²L-³G-⁴A-⁵E-⁶Q/H-⁷X-⁸T-⁹X-¹⁰X-¹¹I/V-¹²I-¹³X-¹⁴R | 36 |
| ¹S-²L-³G-⁴A-⁵E-⁶Q/H-⁷X-⁸A-⁹E-¹⁰X-¹¹I/V-¹²I-¹³X-¹⁴R | 37 |
| ¹S-²L-³G-⁴A-⁵E-⁶Q/H-⁷X-⁸X-⁹A-¹⁰X-¹¹I/V-¹²I-¹³X-¹⁴R | 38 |
| ¹S-²L-³G-⁴A-⁵E-⁶Q/H-⁷X-⁸X-⁹X-¹⁰S-¹¹I/V-¹²I-¹³X-¹⁴R | 39 |
| ¹S-²L-³G-⁴A-⁵E-⁶Q/H-⁷X-⁸X-⁹X-¹⁰A-¹¹I/V-¹²I-¹³X-¹⁴R | 40 |
| ¹S-²L-³G-⁴A-⁵E-⁶Q/H-⁷X-⁸X-⁹X-¹⁰S-¹¹I/V-¹²I-¹³H-¹⁴R | 41 |
| ¹S-²L-³G-⁴A-⁵E-⁶Q/H-⁷X-⁸X-⁹X-¹⁰A-¹¹I/V-¹²I-¹³A-¹⁴R | 42 |
| ¹S-²L-³G-⁴A-⁵E-⁶Q/H-⁷R-⁸X-⁹X-¹⁰X-¹¹I/V-¹²I-¹³X-¹⁴R | 43 |
| ¹S-²L-³G-⁴A-⁵E-⁶Q/H-⁷R-⁸T-⁹X-¹⁰X_¹¹I/V-¹²I-¹³X-¹⁴R | 44 |
| ¹S-²L-³G-⁴A-⁵E-⁶Q/H-⁷R-8A-⁹E-¹⁰X-¹¹I/V-¹²I-¹³X-¹⁴R | 45 |
| ¹S-²L-³G-⁴A-⁵E-⁶Q/H-⁷R-⁸X-⁹A-¹⁰X-¹¹I/V-¹²I-¹³X-¹⁴R | 46 |
| 1S-²L-³G-⁴A-⁵E-⁶Q/H-⁷R-⁸X-⁹X-¹⁰S-¹¹I/V-¹²I-¹³X-¹⁴R | 47 |
| ¹S-²L-³G-⁴A-⁵E-⁶Q/H-⁷S-⁸X-⁹X-¹⁰S-¹¹I/V-¹²I-¹³X-¹⁴R | 48 |
| ¹S-²L-³G-⁴A-⁵E-⁶Q/H-⁷S-⁸X-⁹X-¹⁰S-¹¹I/V-¹²I-¹³H-¹⁴R | 49 |
| ¹S-²L-³G-⁴A-⁵E-⁶Q/H-⁷S-⁸X-⁹X-¹⁰S-¹¹I/V-¹²I-¹³A-¹⁴R | 50 |
| ¹S-²L-³G-⁴A-⁵E-⁶Q/H-⁷S-⁸T-⁹X-¹⁰X-¹¹I/V-¹²I-¹³X-¹⁴R | 51 |
| ¹S-²L-³G-⁴A-⁵E-⁶Q/H-⁷S-⁸T-⁹E-¹⁰X-¹¹I/V-¹²I-¹³X-¹⁴R | 52 |
| ¹S-²L-³G-⁴A-⁵E-⁶Q/H-⁷S-⁸T-⁹S-¹⁰X-¹¹I/V-¹²I-¹³X-¹⁴R | 53 |
| ¹S-²L-³G-⁴A-⁵E-⁶Q/H-⁷S-⁸T-⁹X-¹⁰S-¹¹I/V-¹²I-¹³X-¹⁴R | 54 |
| ¹S-²L-³G-⁴A-⁵E-⁶Q/H-⁷S-⁸T-⁹X-¹⁰S-¹¹I/V-¹²I-¹³X-¹⁴R | 55 |
| ¹S-²L-³G-⁴A-⁵E-⁶Q/H-⁷S-⁸T-⁹X-¹⁰S-¹¹I/V-¹²I-¹³H-¹⁴R | 56 |
| ¹S-²L-³G-⁴A-⁵E-⁶Q/H-⁷S-⁸T-⁹X-¹⁰S-¹¹I/V-¹²I-¹³A-¹⁴R | 57 |
| ¹S-²L-³G-⁴A-⁵E-⁶Q/H-⁷S-⁸T-⁹E-¹⁰S-¹¹I/V-¹²I-¹³X-¹⁴R | 58 |
| ¹S-²L-³G-⁴A-⁵E-⁶Q/H-⁷S-⁸T-⁹E-¹⁰S-¹¹I/V-¹²I-¹³X-¹⁴R | 59 |
| ¹S-²L-³G-⁴A-⁵E-⁶Q/H-⁷S-⁸T-⁹E-¹⁰X-¹¹I/V-¹²I-¹³H-¹⁴R | 60 |
| ¹S-²L-³G-⁴A-⁵E-⁶Q/H-⁷S-⁸T-⁹E-¹⁰X-¹¹I/V-¹²I-¹³A-¹⁴R | 61 |
| ¹S-²L-³G-⁴A-⁵E-⁶Q/H-⁷R-⁸T-⁹E-¹⁰S-¹¹I/V-¹²I-¹³X-¹⁴R | 62 |
| ¹S-²L-³G-⁴A-⁵E-⁶Q/H-⁷R-⁸T-⁹E-¹⁰S-¹¹I/V-¹²I-¹³A-¹⁴R | 63 |
| ¹S-²L-³G-⁴A-⁵E-⁶Q/H-⁷R-⁸T-⁹X-¹⁰S-¹¹I/V-¹²I-¹³H-¹⁴R | 64 |
| ¹S-²L-³G-⁴A-⁵E-⁶Q/H-⁷R-⁸T-⁹A-¹⁰S-¹¹I/V-¹²I-¹³H-¹⁴R | 65 |
| ¹S-²L-³G-⁴A-⁵E-⁶Q/H-⁷R-⁸X-⁹E-¹⁰S-¹¹I/V-¹²I-¹³H-¹⁴R | 66 |
| ¹S-²L-³G-⁴A-⁵E-⁶Q/H-⁷R-⁸A-⁹E-¹⁰S-¹¹I/V-¹²I-¹³H-¹⁴R | 67 |
| ¹S-²L-³G-⁴A-⁵E-⁶Q/H-⁷X-⁸T-⁹E-¹⁰S-¹¹I/V-¹²I-¹³H-¹⁴R | 68 |
| ¹S-²L-³G-⁴A-⁵E-⁶Q/H-⁷A-⁸T-⁹E-¹⁰S-¹¹I/V-¹²I-¹³H-¹⁴R | 69 |
| ¹S-²L-³G-⁴A-⁵X-⁶Q/H-⁷R-⁸T-⁹E-¹⁰S-¹¹I/V-¹²I-¹³H-¹⁴R | 70 |
| ¹S-²L-³G-⁴A-⁵A-⁶Q/H-⁷R-⁸T-⁹E-¹⁰S-¹¹I/V-¹²I-¹³H-¹⁴R | 71 |
| ¹S-²L-³X-⁴A-⁵E-⁶Q/H-⁷R-⁸T-⁹E-¹⁰S-¹¹I/V-¹²I-¹³H-¹⁴R | 72 |
| ¹S-²L-³A-⁴A-⁵E-⁶Q/H-⁷R-⁸T-⁹E-¹⁰S-¹¹I/V-¹²I-¹³H-¹⁴R | 73 |
| ¹S-²X-³G-⁴A-⁵E-⁶Q/H-⁷R-⁸T-⁹E-¹⁰S-¹¹I/V-¹²I-¹³H-¹⁴R | 74 |
| ¹S-²A-³G-⁴A-⁵E-⁶Q/H-⁷R-⁸T-⁹E-¹⁰X-¹¹I/V-¹²I-¹³H-¹⁴R | 75 |

PDSP peptides of the invention may be chemically synthesized or expressed using protein/peptide expression systems. These PDSP peptides may be used in a pharmaceutical composition for the prevention and/or treatment of osteoarthritis. The pharmaceutical composition may comprise any pharmaceutically acceptable excipient, and the pharmaceutical composition may be formulated in a form suitable for administration, such as topical application, oral application, injection, etc. Various formulations for such applications are known in the art and can be used with embodiments of the invention.

PEDF derivatives of the invention, e.g., PDSP 29-mer, 29 amino acids in length, stimulated proliferation of acinar progenitor cells as well as lipogenesis, which was evidenced by higher number of p63 positive basal cells, more Oil Red O (ORO) staining in whole mount and cryosection specimens of PDSP-treated old mice, as compared with blank-treated mice. The 29-mer also improved tear film stability of old mice.

Results described in this invention show that PEDF has a higher expression in acinar undifferentiated progenitor cells than in the differentiated meibocyte. The expression of PEDF protein in MGs declined in old mice, with a significant decreased cell cycle and p63 labeling of acinar progenitor cells. Other results show that the levels of PEDF expression were reduced with increasing ages in the choroid/RPE complex and skin. The decline of PEDF proteins in various tissues in normal aging process may be critical for age-related diseases.

Results from our studies also show that injections of PDSP (e.g., the 29-mer) directly into young and old mice resulted in the proliferation of basal acinar cells at 24 hours. At day 5, old mice exhibited significant difference of cellular proliferation between 29-mer and DMSO injections, but young mice did not. In young mice, the intrinsic PEDF level was higher than old mice, and adding 29-mer may reach a steady-state PEDF concentration and receptor occupancy. Therefore, no significant increase was detected in young mice.

Meibomian gland is a modified sebaceous gland with holocrine differentiation. Differentiation of sebocytes is strongly associated with enhanced lipid synthesis and accumulation in the cells. Our study revealed that PDSP not only exerts the promitotic effect on acinar progenitor cells, but also enhances acinar differentiation. The signaling pathway in PEDF-mediated lipogenesis possibly involves PPARγ signaling. Thus, PEDF may promote acinar differentiation through regulating PPARγ.

MGD has been shown to be associated with proinflammatory cytokines IL-1α and mature IL-1β in ocular surface.³⁷ PEDF is known for its anti-inflammatory activity.¹² PEDF has been demonstrated to block IL-1β by suppressing activation of inflammatory mediator c-Jun N-terminal kinase in human hepatocytes.⁴⁰ Thus, PEDF may improve symptoms of MGD patients through ameliorating inflammatory proteins in the ocular surface. Results presented herein show that 29-mer has no effect on tear secretion but can increase the production of lipids and increases tear film stability, as evidenced by increased TBUT.

In sum, results reported herein indicate that PEDF peptide derivative can promote acinar progenitor cell proliferation. The direct stimulation of the proliferation of acinar progenitor cells, and the improved lipogenesis and tear film stability in vivo suggest PEDF peptide derivative as potential remedy for MGD.

Embodiments of the invention will be illustrated with the following specific examples. In specific examples, the 29mer (SEQ ID NO:1) are used. However, other PDSP (e.g., 14mer, SEQ ID NO:2 or SEQ ID NO:3, etc.) can also be used to achieve the same results. One skilled in the art would appreciate that these examples are for illustration only and that variations and modifications are possible without departing from the scope of the invention.

### Chemicals and Antibodies

Antibodies used in this study were anti-PEDF antibodies (sc-25594, Santa Cruz Biotechnology, CA), BrdU (GTX42641, GeneTex, San Antonio, Tex.) and p63 (mab4135, Millipore, Billerica, Mass.). The 29-mer (Ser⁹³-Thr¹²¹) and 18-mer (Glu⁹⁷-Ser¹¹⁴; control peptide) were synthesized, modified by acetylation of the NH2 termini and amidation of the COOH termini for stability, and characterized by mass spectrometry (>90% purity) at GenScript (Piscataway, N.J.).

### Animals and Treatment

Twelve to fifteen (12-15) months old C57BL/6 mice and 4-8 months old C57BL/6 mice were used. These mice were kept in standard pathogen-free environment at 24° C.±1° C., relatively humidity 60%±10%. All procedures were approved by the Mackay Memorial Hospital Review Board for animal investigation and were conducted in accordance with the ARVO statement for the Use of Animals in Ophthalmic and Vision Research. Mice were anesthetized by an intraperitoneal injection of a mixture of zoletil (6 mg/kg) and xylazine (3 mg/kg). One drop of 0.5% proparacaine hydrochloride (Alcaine; Alcon, Fort Worth, Tex., USA) was given before any ocular procedure.

The 29-mer was reconstituted in DMSO to a final concentration 100 μM. A separated dose of 10 μl of 29-mer (100 μM) mixed with 90 μl of phosphate-buffered saline (PBS) was injected into the upper and lower conjunctival fornix. 10 μl of DMSO mixed with 90 μl of PBS served as a control. To evaluate the effect of 29-mer on tear film break-up time (TBUT) and tear secretion of old mice, subconjunctival injection of 29-mer was introduced weekly till one month and then followed up for two months.

At one month, upper eyelids were subjected to whole mount Oil Red O (ORO) staining. The size of MG tissue in whole mount was quantified using the color range selection and histogram tool with a computer-assisted image analyzer (Adobe Photoshop 7.0) and was calculated in Pixels.

### Tear Film Break-Up Time

To avoid the reduced lipids secretion resulting from no eye blinking under long-term anesthesia, Tear break-up time (TBUT)^{19,20} was performed immediately after mice were anesthetized. 1.5 μL of 0.1% topical fluorescein (Fluor-I-Strip; Ayerst Laboratories, Philadelphia, Pa., USA) was dropped onto the ocular surface. After three compulsory blinks, TBUT was recorded in milliseconds under a slit-lamp with a blue-free barrier filter. Three measurements were taken from each eye. TBUT was taken at similar time point of the day (2-3 PM) in the standard environment by one ophthalmologist who was blinded to the treatment groups.

### Eyelid Whole Mount

After removing hairs, fresh mouse eyelids were collected and immediately fixed with 4% paraformaldehyde overnight, and rinsed with PBS.⁴ Oil-Red-O (ORO) solution was prepared by mixing the stock solution (300 mg ORO powder in 100 ml of 99% isopropanol) and filtered. Eyelids were placed in 60% 2-propanol for 15 minutes, stained with ORO solution for 30 minutes and then de-stained with 60% 2-propanol for 15-20 minutes to achieve optimal lipid staining.⁴ Then, the eyelids were mounted and photographed with the use of a microscope.

### Meiboscale

MGs of young and old mice were graded according to the meiboscale for meibography images.²¹ Briefly, the MG atrophy was denoted as grade 0 when there was no area of loss, grade 1 when area of loss was <25%, grade 2 when area of loss was 25%-50%, grade 3 when area of loss was 51%-75%, and grade 4 when area of loss was 75%. The scores of MGs was as follows and analyzed; grade 0, 5; grade 1, 4; grade 2, 3; grade 3, 2; grade 4,1.

### Oil Red O for Lipids

Eyelid tissues were embedded in OCT and sectioned at 8 μm of thickness. Frozen sections were placed in 60% 2-propanol for 1 minute, stained with filtered ORO solution for 15 minutes, rinsed with PBS and counterstained with hematoxylin.⁴

### 5-Bromo-2′-Deoxyuridine Incorporation Assay

After subconjunctival injection of 29-mer or DMSO, 5-Bromo-2′deoxyuridine (BrdU) 0.1 mg/g of body weight was injected intraperitoneally. The upper eyelids were harvested at 24 hours to evaluate the proliferation of acinar progenitor cells. To study the mitosis of cells, mice were administered by intraperitoneal injections of BrdU daily for 3 days, and the upper eyelids were harvested at day 5. Before performing immunohistochemistry of BrdU, slides were treated with 1 N HCL at 95° C. for 20 minutes.

### Measurement of Tear Volume

The amount of tears was measured with the phenol red thread tear test using ZONE-QUICK cotton threads (Yokota, Tokyo, Japan).^{19,20} After general anesthesia, the lower eyelid was pulled down slightly, and a 1 mm portion of the thread was placed on the palpebral conjunctiva at the point ⅓ of the distance from the lateral canthus. Each eye was tested with the eyes open for 1 minute. The red portion of the thread is measured in millimeters.

### Immunohistochemistry

Immunohistochemistry (IHC) was performed as previously described and modified.²² Formalin-fixed, paraffin-embedded, mice specimens were deparaffinized in xylene and rehydrated in a graded series of ethanol concentrations. Slides were blocked with 10% goat serum for 60 minutes and then incubated with primary antibody against BrdU (1:800 dilution), PEDF (1:50), or p63 (1:200) overnight at 4° C. The slides were subsequently incubated with the appropriate peroxidase-labeled goat immunoglobulin (1:500 dilution; Chemicon, Temecula, Calif.) for 20 minutes and then incubated with chromogen substrate (3,3-diaminobenzidine) for 2 minutes before counterstaining with hematoxylin. Quantification was estimated based on high quality images captured using a Pannoramic digital slide scanners (3Dhistech Ltd. Budapest, Hungary).

### PEDF Staining Grading

PEDF expression was graded according to the following: (A), weak staining of whole acini: 0; strong staining of whole acinar: 1; (B), no trend of stronger staining in basal cytoplasm than other area of the same acini: 0; weak staining of basal acinar cytoplasm but stronger than other area: 1; moderate staining of basal acinar cytoplasm and stronger than other area: 2; strong staining of basal acinar cytoplasm and stronger than other area: 3; (C), No expression in basal cell nucleus: 0, <50% basal cells nuclei stained positive for PEDF: 1; >50% basal cells nuclei stained positive for PEDF: 2. The (A)+(B)+(C) scores were summed, and the total scores can range from 0 to 6.

### Statistical Analysis

Results were presented as mean±SD. SPSS version 18.0 (SPSS Inc., Chicago, Ill., USA) were used for statistical analysis. Mann-Whitney test was used for statistical comparisons. A value of P<0.05 was considered statistically significant.

### MG Atrophy in Old Mice

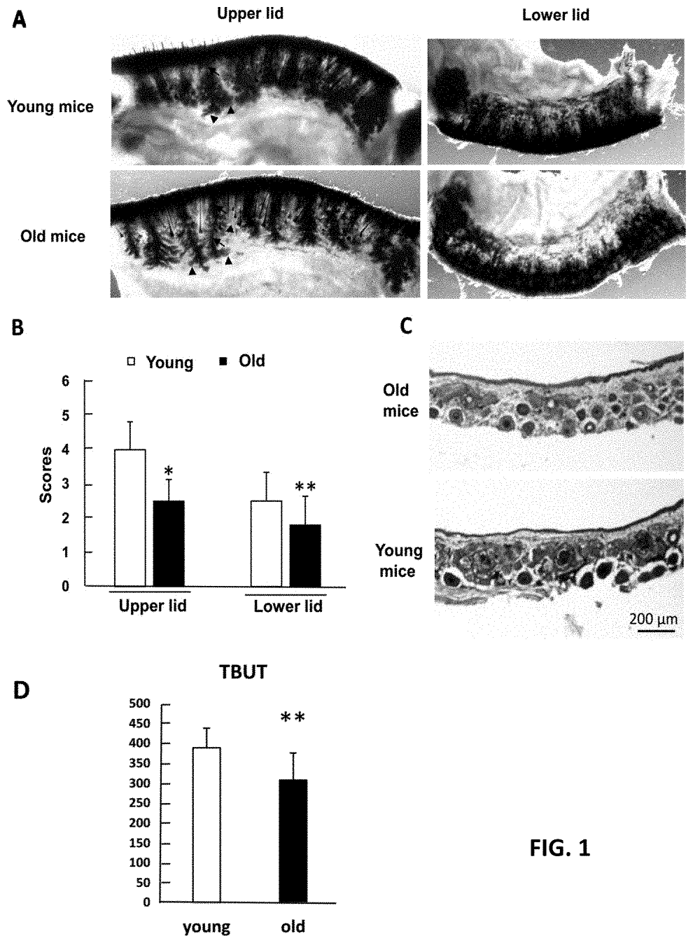

In old mice, reduction in MG acinar sizes was found in ORO staining of whole mount (FIG. 1A). The scores of MGs in upper eyelids of young and old mice were 4±0.82, 2.5±0.63 (P=0.015), respectively. The scores of MGs in lower eyelids of young and old mice were 2.5±0.84, 1.8±0.84 (P=0.006), respectively (FIG. 1B). The finding of cross sections of the eyelids stained with ORO was correlated the morphological change detected by whole mounts (FIG. 1C). In old mice, reduction of acinar size around a central duct was visible in cryosections stained with ORO. Tear break-up time (TBUT) of old mice was 317.36±119.76, which was significant shorter than 389.04±49.18 in young mice (P<0.001) (FIG. 1D). These results indicate that old mice have significant MG atrophy and tear film instability.

### Reduction of PEDF Protein Expression in MGs Acinar of Old Mice

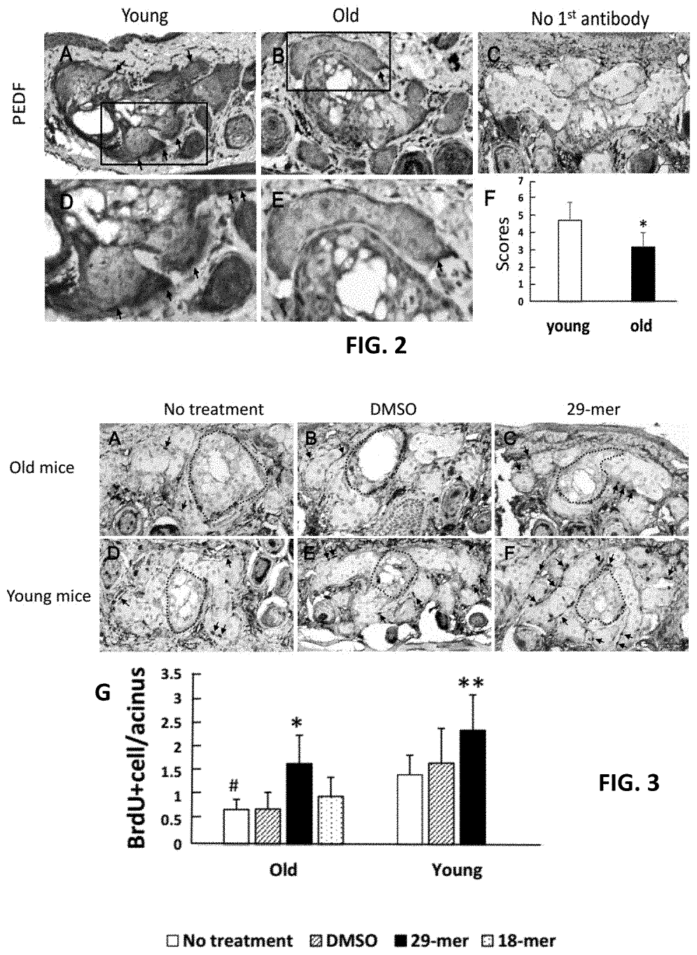

To observe the distribution of PEDF protein in whole acinar, cross sections⁴ of acini of upper eyelids were studied by IHC. The results showed that PEDF expressed in the nucleus of progenitor cells or early differentiated meibocytes near the progenitor cells (FIGS. 2A, B, D, and E). Further, the intensity of PEDF expression was stronger in the cytoplasm at acinar base than proximal end near the ductal tissue (FIG. 2D). The trend of higher expression of PEDF protein in the acinar basal cytoplasm was less prominent in old mice (FIG. 2E). In old mice the intensity of PEDF expression in acini tissue, including the nucleus of progenitor cells, decreased significantly as compared with young mice. The overall scores of PEDF protein expression, shown in FIG. 2F, was decreased in old mice, as compared with that of young mice (3.17±0.83 vs 4.72±1.04, P<0.001).

PEDF peptide promotes proliferation of acinar progenitor cells Decrease in cell cycling of MG acini was found in aging mice.⁷ To evaluate the promitotic effect of PDSP on MG acinar size, mice were intraperitoneally injected with BrdU and euthanized at 24 hours after treatment. We found that at 24 hours, BrdU-positive cells were all acinar progenitor cells, locating at the base of acini. Without any treatment, young mice had more BrdU positive cells per acinus, as compared with old mice (FIGS. 3A and B, 1.44±0.40 vs. 0.73±0.21, P=0.001). The proliferation rate of no treatment group was similar to that of DMSO-treated group in young or old mice (FIGS. 3A, B, D, and E). The 29-mer peptide was able to increase the number of proliferating cells in young mice, as compared with DMSO (FIG. 3F, 2.35±0.73 vs 1.68±0.71 P=0.041). There was also an increase in the number of BrdU-positive cells in PDSP-treated old mice, as compared with DMSO group (FIG. 3C, 1.67±0.58 vs. 0.74±0.34 cells/per acinus, P=0.002). A control peptide 18-mer showed no effect on cell proliferation in old mice (FIG. 3G, 1.00±0.39, p=0.156 compared with DMSO).

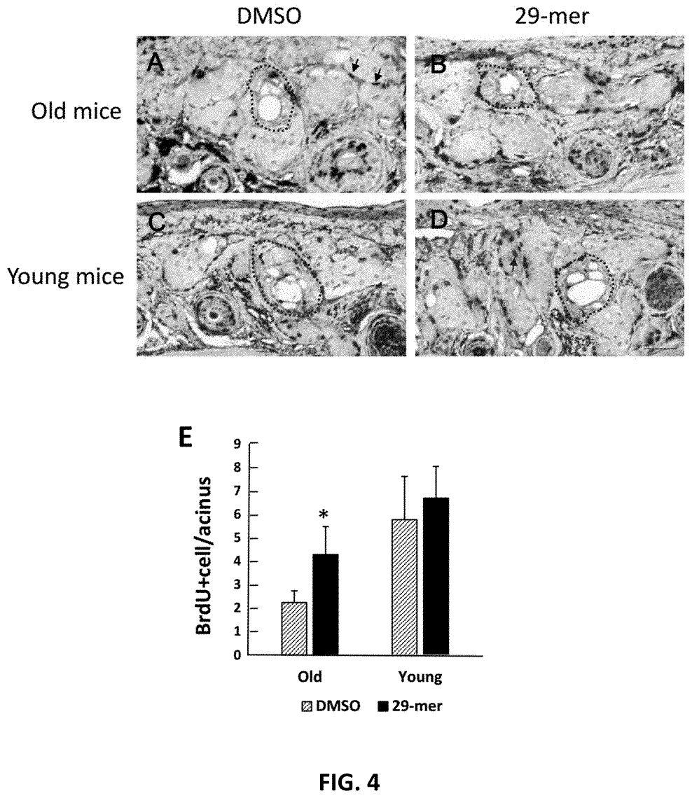

To evaluate the impact on MG homeostasis, mice were intraperitoneally injected with BrdU for 3 days and euthanized at day 5. The BrdU pulse-labeling assay indicated continuously increasing cell proliferation from 24 hours to day 5, and PDSP-treated old mice revealed higher proliferation than DSMO-treated old mice (FIGS. 4A, B, and E, 4.29±1.19 vs. 2.24±0.50, P<0.001). In contrast, there was no difference of cell proliferation between PDSP-treated and DMSO-treated young mice at day 5 (FIGS. 4C and D, 6.70±1.35 vs 5.78±1.84, P=0.233). Of note, some meibocytes were positive for BrdU staining at day 5 (FIG. 4D). The above results indicated that 29-mer, which advanced acinar progenitor cell proliferation, did completely block the differentiation of meibocytes.

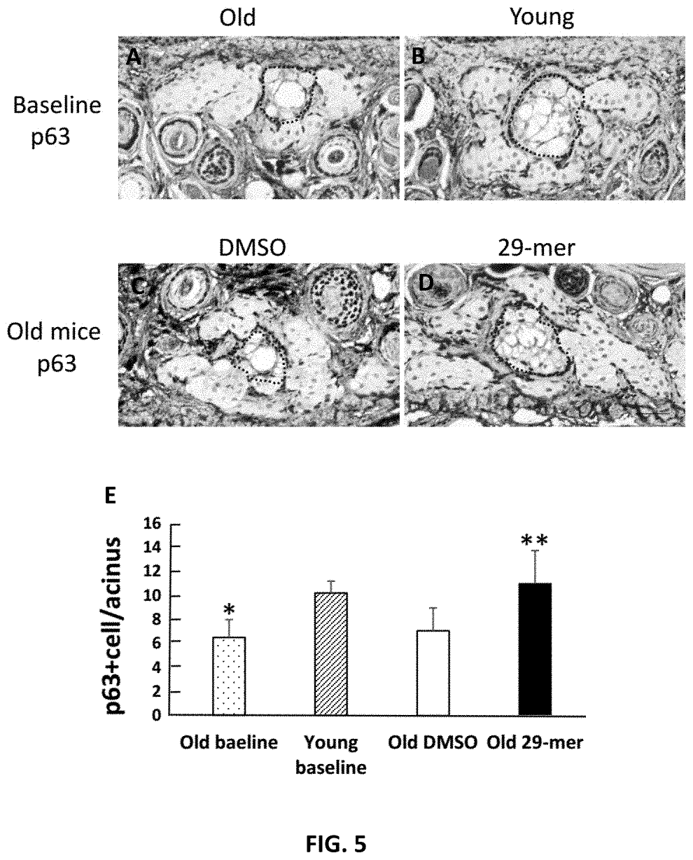

We further investigate the number of acinar progenitor cells using p63 as a marker.⁴ The number of p63-expressing cells significantly reduced in aging mice, as compared with young mice (FIGS. 5A and B, 6.51±1.48 vs 10.21±0.98, P<0.001). 29-mer increased the number of p63-expressing cells of aging mice to a level which was similar to young mice (FIG. 5D, 10.98±2.75). Besides, DMSO had no effect on augmenting acinar progenitor cells (FIG. 5C, 7.06±1.9, P<0.001 compared with 29-mer).

### PEDF Peptide Improves TBUT and Lipogenesis

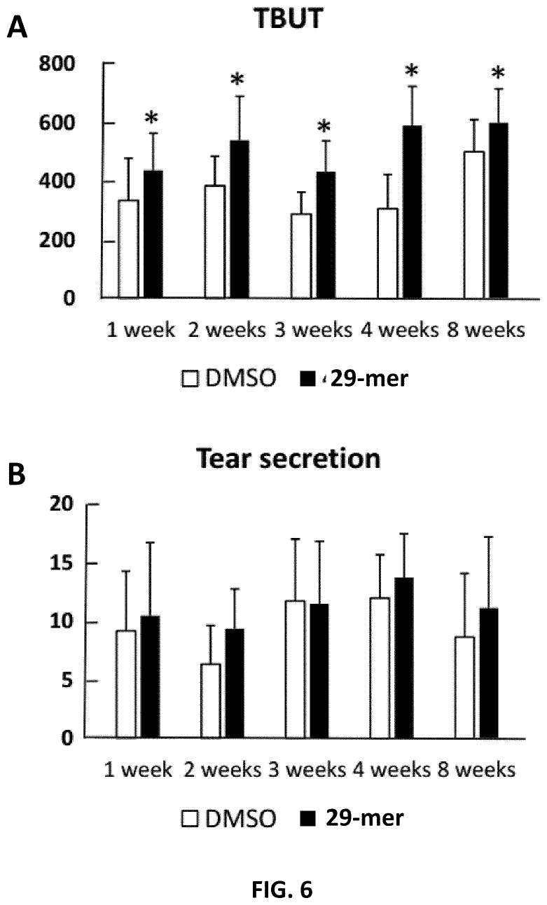

To evaluate the effect of 29-mer on lipids (meibum) formation, subconjunctival injection of 29-mer was introduced weekly up to 4 weeks. TBUT and phenol red thread tear secretion test were performed at 1, 2, 3, 4 and 8 weeks (FIG. 6A). TBUT in 29-mer treated mice was significantly longer than that in control from week 1 to weeks 4. The difference was statistically significant up to 8 weeks. Tear secretion test was invariant between two groups (FIG. 6B). These results suggest that the increased TBUT was due to an improvement of lipid layer.

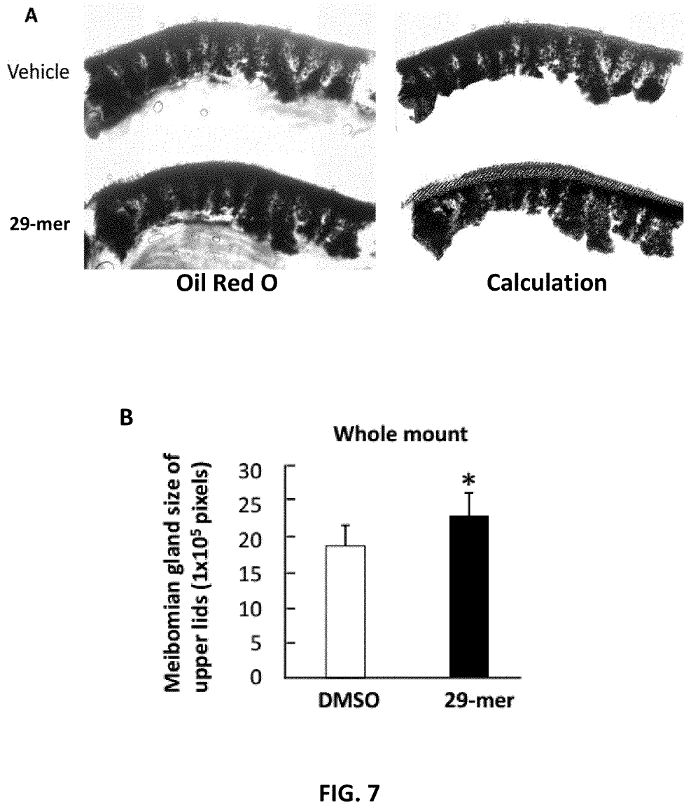

We further investigated the lipids secreted by MGs. Lipid (meibum) production by MGs in the upper eyelids was assessed by eyelid whole mounts stained with ORO. An increase in MG acinar size was visible after 29-mer treatment compared to DMSO (FIG. 7A, 2347530±34986.4 vs 1921689±299347.1 pixels/eyelid, P=0.048) Cross sections of the eyelids stained with ORO showed more overall ORO staining within the cytoplasm of differentiating meibocytes in PDSP-treated mice (FIG. 7B).

While the above examples use the 29-mer to illustrate embodiments of the invention, the core peptide that has the activity is a 14-mer. As noted above, alanine scanning identified the essential residues in the 14-mer and substitutions at the non-essential residues are tolerated. These other variants of the PDSP can also be used with embodiments of the invention.

Embodiments of the invention have been illustrated with a limited number of examples. One skilled in the art would appreciate that variations and modifications are possible without departing from the scope of the invention. Therefore, the scope of the invention should only be limited by the accompanied claims.

### REFERENCES

1. Finis D, Hayajneh J, Konig C, Borrelli M, Schrader S, Geerling G. Evaluation of an automated thermodynamic treatment (LipiFlow®) system for meibomian gland dysfunction: a prospective, randomized, observer-masked trial. Ocul Surf 2014; 12(2):146-154.
2. Liu R, Rong B, Tu P, et al. Analysis of Cytokine Levels in Tears and Clinical Correlations After Intense Pulsed Light Treating Meibomian Gland Dysfunction. Am J Ophthalmol 2017; 183:81-90.
3. Knop E, Knop N, Millar T, Obata H, Sullivan D A. The international workshop on meibomian gland dysfunction: report of the subcommittee on anatomy, physiology, and pathophysiology of the meibomian gland. Invest Ophthalmol Vis Sci 2011; 52(4):1938-1978.
4. Reneker L W, Wang L, Irlmeier R T, Huang A J W. Fibroblast Growth Factor Receptor 2 (FGFR2) Is Required for Meibomian Gland Homeostasis in the Adult Mouse. Invest Ophthalmol Vis Sci 2017; 58(5):2638-2646.
5. Jester J V, Parfitt G J, Brown D J. Meibomian gland dysfunction: hyperkeratinization or atrophy? BMC Ophthalmol 2015; 15 Suppl 1:156.
6. Ong B L, Hodson S A, Wigham T, Miller F, Larke J R. Evidence for keratin proteins in normal and abnormal human meibomian fluids. Curr Eye Res 1991; 10(12):1113-1119.
7. Nien C J, Paugh J R, Massei S, Wahlert A J, Kao W W, Jester J V. Age-related changes in the meibomian gland. Exp Eye Res 2009; 89(6):1021-1027.
8. Nien C J, Massei S, Lin G, et al. Effects of age and dysfunction on human meibomian glands. Arch Ophthalmol 2011; 129(4):462-469.
9. Suhalim I L, Parfitt G J, Xie Y, et al. Effect of desiccating stress on mouse meibomian gland function. Ocul Surf 2014; 12(1):59-68.
10. Tombran-Tink J, Johnson L V. Neuronal differentiation of retinoblastoma cells induced by medium conditioned by human RPE cells. Invest Ophthalmol Vis Sci 1989; 30(8):1700-1707.
11. Steele F R, Chader G J, Johnson L V, Tombran-Tink J. Pigment epithelium-derived factor: neurotrophic activity and identification as a member of the serine protease inhibitor gene family. Proc Natl Acad Sci USA 1993; 90(4):1526-1530.
12. Sagheer U, Gong J, Chung C. Pigment Epithelium-Derived Factor (PEDF) is a Determinant of Stem Cell Fate: Lessons from an Ultra-Rare Disease. J Dev Biol 2015; 3(4):112-128.
13. He X, Cheng R, Benyajati S, Ma J X. PEDF and its roles in physiological and pathological conditions: implication in diabetic and hypoxia-induced angiogenic diseases. Clin Sci (Lond) 2015; 128(11):805-823.
14. Tombran-Tink J, Mazuruk K, Rodriguez I R, et al. Organization, evolutionary conservation, expression and unusual Alu density of the human gene for pigment epithelium-derived factor, a unique neurotrophic serpin. Mol Vis 1996; 2:11.
15. Ho T C, Chiang Y P, Chuang C K, et al. PEDF-derived peptide promotes skeletal muscle regeneration through its mitogenic effect on muscle progenitor cells. Am J Physiol Cell Physiol 2015; 309(3):C159-168.
16. Ho T C, Chen S L, Wu J Y, et al. PEDF promotes self-renewal of limbal stem cell and accelerates corneal epithelial wound healing. Stem Cells 2013; 31(9):1775-1784.
17. Yeh S I, Ho T C, Chen S L, et al. Pigment Epithelial-Derived Factor Peptide Facilitates the Regeneration of a Functional Limbus in Rabbit Partial Limbal Deficiency. Invest Ophthalmol Vis Sci 2015; 56(4):2126-2134.
18. Yeh S I, Ho T C, Chen S L, et al. Pigment Epithelial-Derived Factor Peptide Regenerated Limbus Serves as Regeneration Source for Limbal Regeneration in Rabbit Limbal Deficiency. Invest Ophthalmol Vis Sci 2016; 57(6):2629-2636.
19. Lin Z, Liu X, Zhou T, et al. A mouse dry eye model induced by topical administration of benzalkonium chloride. Mol Vis 2011; 17:257-264.
20. Wang Y C, Li S, Chen X, et al. Meibomian Gland Absence Related Dry Eye in Ectodysplasin A Mutant Mice. Am J Pathol 2016; 186(1):32-42.
21. Pult H, Nichols J J. A review of meibography. Optom Vis Sci 2012; 89(5):E760-769.
22. Ho T C, Chen S L, Shih S C, et al. Pigment epithelium-derived factor is an intrinsic antifibrosis factor targeting hepatic stellate cells. Am J Pathol 2010; 177(4):1798-1811.
23. Sarojini H, Estrada R, Lu H, et al. PEDF from mouse mesenchymal stem cell secretome attracts fibroblasts. J Cell Biochem 2008; 104(5):1793-1802.
24. Li F, Song N, Tombran-Tink J, Niyibizi C. Pigment epithelium-derived factor enhances differentiation and mineral deposition of human mesenchymal stem cells. Stem Cells 2013; 31(12):2714-2723.
25. Gattu A K, Swenson E S, Iwakiri Y, et al. Determination of mesenchymal stem cell fate by pigment epithelium-derived factor (PEDF) results in increased adiposity and reduced bone mineral content. FASEB J 2013; 27(11):4384-4394.
26. Steinle J J, Sharma S, Chin V C. Normal aging involves altered expression of growth factors in the rat choroid. J Gerontol A Biol Sci Med Sci 2008; 63(2):135-140.
27. Francis M K, Appel S, Meyer C, Balin S J, Balin A K, Cristofalo V J. Loss of EPC-1/PEDF expression during skin aging in vivo. J Invest Dermatol 2004; 122(5):1096-1105.
28. Morris D L, Rui L. Recent advances in understanding leptin signaling and leptin resistance. Am J Physiol Endocrinol Metab 2009; 297(6):E1247-1259.
29. Knauer D J, Wiley H S, Cunningham D D. Relationship between epidermal growth factor receptor occupancy and mitogenic response. Quantitative analysis using a steady state model system. J Biol Chem 1984; 259(9):5623-5631.
30. Hong I, Lee M H, Na T Y, Zouboulis C C, Lee M O. LXRalpha enhances lipid synthesis in SZ95 sebocytes. J Invest Dermatol 2008; 128(5):1266-1272.
31. Hwang H S, Parfitt G J, Brown D J, Jester J V. Meibocyte differentiation and renewal: Insights into novel mechanisms of meibomian gland dysfunction (MGD). Exp Eye Res 2017; 163:37-45.
32. Ho T C, Chen S L, Yang Y C, et al. Cytosolic phospholipase A2-{alpha} is an early apoptotic activator in PEDF-induced endothelial cell apoptosis. Am J Physiol Cell Physiol 2009; 296(2):C273-284.
33. Ho T C, Chen S L, Yang Y C, Liao C L, Cheng H C, Tsao Y P. PEDF induces p53-mediated apoptosis through PPAR gamma signaling in human umbilical vein endothelial cells. Cardiovasc Res 2007; 76(2):213-223.
34. Liu Y, Zhu Y, Rannou F, et al. Laminar flow activates peroxisome proliferator-activated receptor-gamma in vascular endothelial cells. Circulation 2004; 110(9):1128-1133.
35. Notari L, Baladron V, Aroca-Aguilar J D, et al. Identification of a lipase-linked cell membrane receptor for pigment epithelium-derived factor. J Biol Chem 2006; 281(49):38022-38037.
36. Yang S L, Chen S L, Wu J Y, Ho T C, Tsao Y P. Pigment epithelium-derived factor induces interleukin-10 expression in human macrophages by induction of PPAR gamma. Life Sci 2010; 87(1-2):26-35.
37. Solomon A, Dursun D, Liu Z, Xie Y, Macri A, Pflugfelder S C. Pro- and anti-inflammatory forms of interleukin-1 in the tear fluid and conjunctiva of patients with dry-eye disease. Invest Ophthalmol Vis Sci 2001; 42(10):2283-2292.
38. Tosato G, Jones K D. Interleukin-1 induces interleukin-6 production in peripheral blood monocytes. Blood 1990; 75(6):1305-1310.
39. Oliveira I C, Sciavolino P J, Lee T H, Vilcek J. Downregulation of interleukin 8 gene expression in human fibroblasts: unique mechanism of transcriptional inhibition by interferon. Proc Natl Acad Sci USA 1992; 89(19):9049-9053.
40. Gattu A K, Birkenfeld A L, Iwakiri Y, et al. Pigment epithelium-derived factor (PEDF) suppresses IL-1beta-mediated c-Jun N-terminal kinase (JNK) activation to improve hepatocyte insulin signaling. Endocrinology 2014; 155(4):1373-1385.
41. Cavaliere R M, Ghirardi F, Tirindelli R. Lacrimal gland removal impairs sexual behavior in mice. Front Neuroanat 2014; 8:101.

What is claimed is:

1. A pharmaceutical composition for use in promoting meibomian gland regeneration or in treating and/or preventing dry eye syndrome, comprising: a PEDF-derived short peptide (PDSP) or a variant of the PDSP, wherein the PDSP comprises residues 93-106 of human pigmented epithelium-derived factor (PEDF), and wherein the variant of the PDSP contains serine-93, alanine-96, glutamine-98, isoleucine-103, isoleucine-104, and arginine 106 of the PDSP and contains one or more amino acid substitutions at other positions, wherein residue location numbers are based on those in the human PEDF.

2. The pharmaceutical composition according to claim 1, wherein the PDSP comprises the sequence of S-X-X-A-X-Q/H-X-X-X-X-I/V-I-X-R (SEQ ID NO:5).

3. The pharmaceutical composition according to claim 1, wherein the PDSP comprises the sequence of SLGAEQRTESIIHR (SEQ ID NO:2).

4. The pharmaceutical composition according to claim 1, wherein the PDSP comprises the sequence of SLGAEQRTESIIHRALYYDLISSPDIHGT (SEQ ID NO:1).

5. The pharmaceutical composition according to claim 1, wherein the PDSP comprises the sequence of the sequences of any one of SEQ ID NO: 6 to 75.


SEQUENCE LISTING (75 sequences)

SEQ ID NO: 1 - protein, 29 aa, Homo sapiens
SLGAEQRTESIIHRALYYDLISSPDIHGT

SEQ ID NO: 2 - protein, 14 aa, Homo sapiens
SLGAEQRTESIIHR

SEQ ID NO: 3 - protein, 29 aa, Mus musculus
SLGAEHRTESVIHRALYYDLITNPDIHST

SEQ ID NO: 4 - protein, 14 aa, Mus musculus
SLGAEHRTESVIHR

SEQ ID NO: 5 - protein, 14 aa, Artificial Sequence
  synthetic
  misc_feature (2)..(3): Xaa can be any naturally occurring amino acid
  misc_feature (5)..(11): Xaa can be any naturally occurring amino acid
  misc_feature (13)..(13): Xaa can be any naturally occurring amino acid
SXXAXXXXXXXIXR

SEQ ID NO: 6 - protein, 14 aa, Artificial Sequence
  synthetic
  misc_feature (3)..(3): Xaa can be any naturally occurring amino acid
  misc_feature (5)..(11): Xaa can be any naturally occurring amino acid
  misc_feature (13)..(13): Xaa can be any naturally occurring amino acid
SLXAXXXXXXXIXR

SEQ ID NO: 7 - protein, 14 aa, Artificial Sequence
  synthetic
  misc_feature (3)..(3): Xaa can be any naturally occurring amino acid
  misc_feature (5)..(11): Xaa can be any naturally occurring amino acid
  misc_feature (13)..(13): Xaa can be any naturally occurring amino acid
SAXAXXXXXXXIXR

SEQ ID NO: 8 - protein, 14 aa, Artificial Sequence
  synthetic
  misc_feature (2)..(2): Xaa can be any naturally occurring amino acid
  misc_feature (5)..(11): Xaa can be any naturally occurring amino acid
  misc_feature (13)..(13): Xaa can be any naturally occurring amino acid
SXGAXXXXXXXIXR

SEQ ID NO: 9 - protein, 14 aa, Artificial Sequence
  synthetic
  misc_feature (2)..(2): Xaa can be any naturally occurring amino acid
  misc_feature (5)..(11): Xaa can be any naturally occurring amino acid
  misc_feature (13)..(13): Xaa can be any naturally occurring amino acid
SXAAXXXXXXXIXR

SEQ ID NO: 10 - protein, 14 aa, Artificial Sequence
  synthetic
  misc_feature (2)..(3): Xaa can be any naturally occurring amino acid
  misc_feature (6)..(11): Xaa can be any naturally occurring amino acid
  misc_feature (13)..(13): Xaa can be any naturally occurring amino acid
SXXAEXXXXXXIXR

SEQ ID NO: 11 - protein, 14 aa, Artificial Sequence
  synthetic
  misc_feature (2)..(3): Xaa can be any naturally occurring amino acid
  misc_feature (6)..(11): Xaa can be any naturally occurring amino acid
  misc_feature (13)..(13): Xaa can be any naturally occurring amino acid
SXXAAXXXXXXIXR

SEQ ID NO: 12 - protein, 14 aa, Artificial Sequence
  synthetic
  misc_feature (2)..(3): Xaa can be any naturally occurring amino acid
  misc_feature (5)..(6): Xaa can be any naturally occurring amino acid
  misc_feature (8)..(11): Xaa can be any naturally occurring amino acid
  misc_feature (13)..(13): Xaa can be any naturally occurring amino acid
SXXAXXRXXXXIXR

SEQ ID NO: 13 - protein, 14 aa, Artificial Sequence
  synthetic
  misc_feature (2)..(3): Xaa can be any naturally occurring amino acid
  misc_feature (5)..(6): Xaa can be any naturally occurring amino acid
  misc_feature (8)..(11): Xaa can be any naturally occurring amino acid
  misc_feature (13)..(13): Xaa can be any naturally occurring amino acid
SXXAXXAXXXXIXR

SEQ ID NO: 14 - protein, 14 aa, Artificial Sequence
  synthetic
  misc_feature (2)..(3): Xaa can be any naturally occurring amino acid
  misc_feature (5)..(7): Xaa can be any naturally occurring amino acid
  misc_feature (9)..(11): Xaa can be any naturally occurring amino acid
  misc_feature (13)..(13): Xaa can be any naturally occurring amino acid
SXXAXXXTXXXIXR

SEQ ID NO: 15 - protein, 14 aa, Artificial Sequence
  synthetic
  misc_feature (2)..(3): Xaa can be any naturally occurring amino acid
  misc_feature (5)..(7): Xaa can be any naturally occurring amino acid
  misc_feature (10)..(11): Xaa can be any naturally occurring amino acid
  misc_feature (13)..(13): Xaa can be any naturally occurring amino acid
SXXAXXXAEXXIXR

SEQ ID NO: 16 - protein, 14 aa, Artificial Sequence
  synthetic
  misc_feature (2)..(3): Xaa can be any naturally occurring amino acid
  misc_feature (5)..(8): Xaa can be any naturally occurring amino acid
  misc_feature (10)..(11): Xaa can be any naturally occurring amino acid
  misc_feature (13)..(13): Xaa can be any naturally occurring amino acid
SXXAXXXXAXXIXR

SEQ ID NO: 17 - protein, 14 aa, Artificial Sequence
  synthetic
  misc_feature (2)..(3): Xaa can be any naturally occurring amino acid
  misc_feature (5)..(9): Xaa can be any naturally occurring amino acid
  misc_feature (11)..(11): Xaa can be any naturally occurring amino acid
  misc_feature (13)..(13): Xaa can be any naturally occurring amino acid
SXXAXXXXXSXIXR

SEQ ID NO: 18 - protein, 14 aa, Artificial Sequence
  synthetic
  misc_feature (2)..(3): Xaa can be any naturally occurring amino acid
  misc_feature (5)..(9): Xaa can be any naturally occurring amino acid
  misc_feature (11)..(11): Xaa can be any naturally occurring amino acid
  misc_feature (13)..(13): Xaa can be any naturally occurring amino acid
SXXAXXXXXAXIXR

SEQ ID NO: 19 - protein, 14 aa, Artificial Sequence
  synthetic
  misc_feature (2)..(3): Xaa can be any naturally occurring amino acid
  misc_feature (5)..(11): Xaa can be any naturally occurring amino acid
SXXAXXXXXXXIHR

SEQ ID NO: 20 - protein, 14 aa, Artificial Sequence
  synthetic
  misc_feature (2)..(3): Xaa can be any naturally occurring amino acid
  misc_feature (5)..(11): Xaa can be any naturally occurring amino acid
SXXAXXXXXXXIAR

SEQ ID NO: 21 - protein, 14 aa, Artificial Sequence
  synthetic
  misc_feature (5)..(11): Xaa can be any naturally occurring amino acid
  misc_feature (13)..(13): Xaa can be any naturally occurring amino acid
SLGAXXXXXXXIXR

SEQ ID NO: 22 - protein, 14 aa, Artificial Sequence
  synthetic
  misc_feature (6)..(11): Xaa can be any naturally occurring amino acid
  misc_feature (13)..(13): Xaa can be any naturally occurring amino acid
SLGAEXXXXXXIXR

SEQ ID NO: 23 - protein, 14 aa, Artificial Sequence
  synthetic
  misc_feature (6)..(11): Xaa can be any naturally occurring amino acid
  misc_feature (13)..(13): Xaa can be any naturally occurring amino acid
SLGAAXXXXXXIXR

SEQ ID NO: 24 - protein, 14 aa, Artificial Sequence
  synthetic
  misc_feature (5)..(6): Xaa can be any naturally occurring amino acid
  misc_feature (8)..(11): Xaa can be any naturally occurring amino acid
  misc_feature (13)..(13): Xaa can be any naturally occurring amino acid
SLGAXXRXXXXIXR

SEQ ID NO: 25 - protein, 14 aa, Artificial Sequence
  synthetic
  misc_feature (5)..(6): Xaa can be any naturally occurring amino acid
  misc_feature (8)..(11): Xaa can be any naturally occurring amino acid
  misc_feature (13)..(13): Xaa can be any naturally occurring amino acid
SLGAXXAXXXXIXR

SEQ ID NO: 26 - protein, 14 aa, Artificial Sequence
  synthetic
  misc_feature (5)..(7): Xaa can be any naturally occurring amino acid
  misc_feature (9)..(11): Xaa can be any naturally occurring amino acid
  misc_feature (13)..(13): Xaa can be any naturally occurring amino acid
SLGAXXXTXXXIXR

SEQ ID NO: 27 - protein, 14 aa, Artificial Sequence
  synthetic
  misc_feature (5)..(7): Xaa can be any naturally occurring amino acid
  misc_feature (10)..(11): Xaa can be any naturally occurring amino acid
  misc_feature (13)..(13): Xaa can be any naturally occurring amino acid
SLGAXXXAEXXIXR

SEQ ID NO: 28 - protein, 14 aa, Artificial Sequence
  synthetic
  misc_feature (5)..(8): Xaa can be any naturally occurring amino acid
  misc_feature (10)..(11): Xaa can be any naturally occurring amino acid
  misc_feature (13)..(13): Xaa can be any naturally occurring amino acid
SLGAXXXXAXXIXR

SEQ ID NO: 29 - protein, 14 aa, Artificial Sequence
  synthetic
  misc_feature (5)..(9): Xaa can be any naturally occurring amino acid
  misc_feature (11)..(11): Xaa can be any naturally occurring amino acid
  misc_feature (13)..(13): Xaa can be any naturally occurring amino acid
SLGAXXXXXSXIXR

SEQ ID NO: 30 - protein, 14 aa, Artificial Sequence
  synthetic
  misc_feature (5)..(9): Xaa can be any naturally occurring amino acid
  misc_feature (11)..(11): Xaa can be any naturally occurring amino acid
  misc_feature (13)..(13): Xaa can be any naturally occurring amino acid
SLGAXXXXXAXIXR

SEQ ID NO: 31 - protein, 14 aa, Artificial Sequence
  synthetic
  misc_feature (5)..(9): Xaa can be any naturally occurring amino acid
  misc_feature (11)..(11): Xaa can be any naturally occurring amino acid
SLGAXXXXXSXIHR

SEQ ID NO: 32 - protein, 14 aa, Artificial Sequence
  synthetic
  misc_feature (5)..(9): Xaa can be any naturally occurring amino acid
  misc_feature (11)..(11): Xaa can be any naturally occurring amino acid
SLGAXXXXXAXIAR

SEQ ID NO: 33 - protein, 14 aa, Artificial Sequence
  synthetic
  misc_feature (6)..(11): Xaa can be any naturally occurring amino acid
  misc_feature (13)..(13): Xaa can be any naturally occurring amino acid
SLGAEXXXXXXIXR

SEQ ID NO: 34 - protein, 14 aa, Artificial Sequence
  synthetic
  misc_feature (6)..(6): Xaa can be any naturally occurring amino acid
  misc_feature (8)..(11): Xaa can be any naturally occurring amino acid
  misc_feature (13)..(13): Xaa can be any naturally occurring amino acid
SLGAEXRXXXXIXR

SEQ ID NO: 35 - protein, 14 aa, Artificial Sequence
  synthetic
  misc_feature (6)..(6): Xaa can be any naturally occurring amino acid
  misc_feature (8)..(11): Xaa can be any naturally occurring amino acid
  misc_feature (13)..(13): Xaa can be any naturally occurring amino acid
SLGAEXAXXXXIXR

SEQ ID NO: 36 - protein, 14 aa, Artificial Sequence
  synthetic
  misc_feature (6)..(7): Xaa can be any naturally occurring amino acid
  misc_feature (9)..(11): Xaa can be any naturally occurring amino acid
  misc_feature (13)..(13): Xaa can be any naturally occurring amino acid
SLGAEXXTXXXIXR

SEQ ID NO: 37 - protein, 14 aa, Artificial Sequence
  synthetic
  misc_feature (6)..(7): Xaa can be any naturally occurring amino acid
  misc_feature (10)..(11): Xaa can be any naturally occurring amino acid
  misc_feature (13)..(13): Xaa can be any naturally occurring amino acid
SLGAEXXAEXXIXR

SEQ ID NO: 38 - protein, 14 aa, Artificial Sequence
  synthetic
  misc_feature (6)..(8): Xaa can be any naturally occurring amino acid
  misc_feature (10)..(11): Xaa can be any naturally occurring amino acid
  misc_feature (13)..(13): Xaa can be any naturally occurring amino acid
SLGAEXXXAXXIXR

SEQ ID NO: 39 - protein, 14 aa, Artificial Sequence
  synthetic
  misc_feature (6)..(9): Xaa can be any naturally occurring amino acid
  misc_feature (11)..(11): Xaa can be any naturally occurring amino acid
  misc_feature (13)..(13): Xaa can be any naturally occurring amino acid
SLGAEXXXXSXIXR

SEQ ID NO: 40 - protein, 14 aa, Artificial Sequence
  synthetic
  misc_feature (6)..(9): Xaa can be any naturally occurring amino acid
  misc_feature (11)..(11): Xaa can be any naturally occurring amino acid
  misc_feature (13)..(13): Xaa can be any naturally occurring amino acid
SLGAEXXXXAXIXR

SEQ ID NO: 41 - protein, 14 aa, Artificial Sequence
  synthetic
  misc_feature (6)..(9): Xaa can be any naturally occurring amino acid
  misc_feature (11)..(11): Xaa can be any naturally occurring amino acid
SLGAEXXXXSXIHR

SEQ ID NO: 42 - protein, 14 aa, Artificial Sequence
  synthetic
  misc_feature (6)..(9): Xaa can be any naturally occurring amino acid
  misc_feature (11)..(11): Xaa can be any naturally occurring amino acid
SLGAEXXXXAXIAR

SEQ ID NO: 43 - protein, 14 aa, Artificial Sequence
  synthetic
  misc_feature (6)..(6): Xaa can be any naturally occurring amino acid
  misc_feature (8)..(11): Xaa can be any naturally occurring amino acid
  misc_feature (13)..(13): Xaa can be any naturally occurring amino acid
SLGAEXRXXXXIXR

SEQ ID NO: 44 - protein, 14 aa, Artificial Sequence
  synthetic
  misc_feature (6)..(6): Xaa can be any naturally occurring amino acid
  misc_feature (9)..(11): Xaa can be any naturally occurring amino acid
  misc_feature (13)..(13): Xaa can be any naturally occurring amino acid
SLGAEXRTXXXIXR

SEQ ID NO: 45 - protein, 14 aa, Artificial Sequence
  synthetic
  misc_feature (6)..(6): Xaa can be any naturally occurring amino acid
  misc_feature (9)..(11): Xaa can be any naturally occurring amino acid
  misc_feature (13)..(13): Xaa can be any naturally occurring amino acid
SLGAEXRAXXXIXR

SEQ ID NO: 46 - protein, 14 aa, Artificial Sequence
  synthetic
  misc_feature (6)..(6): Xaa can be any naturally occurring amino acid
  misc_feature (8)..(8): Xaa can be any naturally occurring amino acid
  misc_feature (10)..(11): Xaa can be any naturally occurring amino acid
  misc_feature (13)..(13): Xaa can be any naturally occurring amino acid
SLGAEXRXAXXIXR

SEQ ID NO: 47 - protein, 14 aa, Artificial Sequence
  synthetic
  misc_feature (6)..(6): Xaa can be any naturally occurring amino acid
  misc_feature (8)..(9): Xaa can be any naturally occurring amino acid
  misc_feature (11)..(11): Xaa can be any naturally occurring amino acid
  misc_feature (13)..(13): Xaa can be any naturally occurring amino acid
SLGAEXRXXSXIXR

SEQ ID NO: 48 - protein, 14 aa, Artificial Sequence
  synthetic
  misc_feature (6)..(6): Xaa can be any naturally occurring amino acid
  misc_feature (8)..(9): Xaa can be any naturally occurring amino acid
  misc_feature (11)..(11): Xaa can be any naturally occurring amino acid
  misc_feature (13)..(13): Xaa can be any naturally occurring amino acid
SLGAEXRXXAXIXR

SEQ ID NO: 49 - protein, 14 aa, Artificial Sequence
  synthetic
  misc_feature (6)..(6): Xaa can be any naturally occurring amino acid
  misc_feature (8)..(9): Xaa can be any naturally occurring amino acid
  misc_feature (11)..(11): Xaa can be any naturally occurring amino acid
SLGAEXRXXSXIHR

SEQ ID NO: 50 - protein, 14 aa, Artificial Sequence
  synthetic
  misc_feature (6)..(6): Xaa can be any naturally occurring amino acid
  misc_feature (8)..(9): Xaa can be any naturally occurring amino acid
  misc_feature (11)..(11): Xaa can be any naturally occurring amino acid
SLGAEXRXXAXIAR

SEQ ID NO: 51 - protein, 14 aa, Artificial Sequence
  synthetic
  misc_feature (6)..(6): Xaa can be any naturally occurring amino acid
  misc_feature (9)..(11): Xaa can be any naturally occurring amino acid
  misc_feature (13)..(13): Xaa can be any naturally occurring amino acid
SLGAEXRTXXXIXR

SEQ ID NO: 52 - protein, 14 aa, Artificial Sequence
  synthetic
  misc_feature (6)..(6): Xaa can be any naturally occurring amino acid
  misc_feature (10)..(11): Xaa can be any naturally occurring amino acid
  misc_feature (13)..(13): Xaa can be any naturally occurring amino acid
SLGAEXRTEXXIXR

SEQ ID NO: 53 - protein, 14 aa, Artificial Sequence
  synthetic
  misc_feature (6)..(6): Xaa can be any naturally occurring amino acid
  misc_feature (10)..(11): Xaa can be any naturally occurring amino acid
  misc_feature (13)..(13): Xaa can be any naturally occurring amino acid
SLGAEXRTAXXIXR

SEQ ID NO: 54 - protein, 14 aa, Artificial Sequence
  synthetic
  misc_feature (6)..(6): Xaa can be any naturally occurring amino acid
  misc_feature (9)..(9): Xaa can be any naturally occurring amino acid
  misc_feature (11)..(11): Xaa can be any naturally occurring amino acid
  misc_feature (13)..(13): Xaa can be any naturally occurring amino acid
SLGAEXRTXSXIXR

SEQ ID NO: 55 - protein, 14 aa, Artificial Sequence
  synthetic
  misc_feature (6)..(6): Xaa can be any naturally occurring amino acid
  misc_feature (9)..(9): Xaa can be any naturally occurring amino acid
  misc_feature (11)..(11): Xaa can be any naturally occurring amino acid
  misc_feature (13)..(13): Xaa can be any naturally occurring amino acid
SLGAEXRTXAXIXR

SEQ ID NO: 56 - protein, 14 aa, Artificial Sequence
  synthetic
  misc_feature (6)..(6): Xaa can be any naturally occurring amino acid
  misc_feature (9)..(9): Xaa can be any naturally occurring amino acid
  misc_feature (11)..(11): Xaa can be any naturally occurring amino acid
SLGAEXRTXSXIHR

SEQ ID NO: 57 - protein, 14 aa, Artificial Sequence
  synthetic
  misc_feature (6)..(6): Xaa can be any naturally occurring amino acid
  misc_feature (9)..(9): Xaa can be any naturally occurring amino acid
  misc_feature (11)..(11): Xaa can be any naturally occurring amino acid
SLGAEXRTXAXIAR

SEQ ID NO: 58 - protein, 14 aa, Artificial Sequence
  synthetic
  misc_feature (6)..(6): Xaa can be any naturally occurring amino acid
  misc_feature (11)..(11): Xaa can be any naturally occurring amino acid
  misc_feature (13)..(13): Xaa can be any naturally occurring amino acid
SLGAEXRTESXIXR

SEQ ID NO: 59 - protein, 14 aa, Artificial Sequence
  synthetic
  misc_feature (6)..(6): Xaa can be any naturally occurring amino acid
  misc_feature (11)..(11): Xaa can be any naturally occurring amino acid
  misc_feature (13)..(13): Xaa can be any naturally occurring amino acid
SLGAEXRTEAXIXR

SEQ ID NO: 60 - protein, 14 aa, Artificial Sequence
  synthetic
  misc_feature (6)..(6): Xaa can be any naturally occurring amino acid
  misc_feature (10)..(11): Xaa can be any naturally occurring amino acid
SLGAEXRTEXXIHR

SEQ ID NO: 61 - protein, 14 aa, Artificial Sequence
  synthetic
  misc_feature (6)..(6): Xaa can be any naturally occurring amino acid
  misc_feature (10)..(11): Xaa can be any naturally occurring amino acid
SLGAEXRTEXXIAR

SEQ ID NO: 62 - protein, 14 aa, Artificial Sequence
  synthetic
  misc_feature (6)..(6): Xaa can be any naturally occurring amino acid
  misc_feature (11)..(11): Xaa can be any naturally occurring amino acid
  misc_feature (13)..(13): Xaa can be any naturally occurring amino acid
SLGAEXRTESXIXR

SEQ ID NO: 63 - protein, 14 aa, Artificial Sequence
  synthetic
  misc_feature (6)..(6): Xaa can be any naturally occurring amino acid
  misc_feature (11)..(11): Xaa can be any naturally occurring amino acid
SLGAEXRTESXIAR

SEQ ID NO: 64 - protein, 14 aa, Artificial Sequence
  synthetic
  misc_feature (6)..(6): Xaa can be any naturally occurring amino acid
  misc_feature (9)..(9): Xaa can be any naturally occurring amino acid
  misc_feature (11)..(11): Xaa can be any naturally occurring amino acid
SLGAEXRTXSXIHR

SEQ ID NO: 65 - protein, 14 aa, Artificial Sequence
  synthetic
  misc_feature (6)..(6): Xaa can be any naturally occurring amino acid
  misc_feature (11)..(11): Xaa can be any naturally occurring amino acid
SLGAEXRTASXIHR

SEQ ID NO: 66 - protein, 14 aa, Artificial Sequence
  synthetic
  misc_feature (6)..(6): Xaa can be any naturally occurring amino acid
  misc_feature (8)..(8): Xaa can be any naturally occurring amino acid
  misc_feature (11)..(11): Xaa can be any naturally occurring amino acid
SLGAEXRXESXIHR

SEQ ID NO: 67 - protein, 14 aa, Artificial Sequence
  synthetic
  misc_feature (6)..(6): Xaa can be any naturally occurring amino acid
  misc_feature (11)..(11): Xaa can be any naturally occurring amino acid
SLGAEXRAESXIHR

SEQ ID NO: 68 - protein, 14 aa, Artificial Sequence
  synthetic
  misc_feature (6)..(7): Xaa can be any naturally occurring amino acid
  misc_feature (11)..(11): Xaa can be any naturally occurring amino acid
SLGAEXXTESXIHR

SEQ ID NO: 69 - protein, 14 aa, Artificial Sequence
  synthetic
  misc_feature (6)..(6): Xaa can be any naturally occurring amino acid
  misc_feature (11)..(11): Xaa can be any naturally occurring amino acid
SLGAEXATESXIHR

SEQ ID NO: 70 - protein, 14 aa, Artificial Sequence
  synthetic
  misc_feature (5)..(6): Xaa can be any naturally occurring amino acid
  misc_feature (11)..(11): Xaa can be any naturally occurring amino acid
SLGAXXRTESXIHR

SEQ ID NO: 71 - protein, 14 aa, Artificial Sequence
  synthetic
  misc_feature (6)..(6): Xaa can be any naturally occurring amino acid
  misc_feature (11)..(11): Xaa can be any naturally occurring amino acid
SLGAAXRTESXIHR

SEQ ID NO: 72 - protein, 14 aa, Artificial Sequence
  synthetic
  misc_feature (3)..(3): Xaa can be any naturally occurring amino acid
  misc_feature (6)..(6): Xaa can be any naturally occurring amino acid
  misc_feature (11)..(11): Xaa can be any naturally occurring amino acid
SLXAEXRTESXIHR

SEQ ID NO: 73 - protein, 14 aa, Artificial Sequence
  synthetic
  misc_feature (6)..(6): Xaa can be any naturally occurring amino acid
  misc_feature (11)..(11): Xaa can be any naturally occurring amino acid
SLAAEXRTESXIHR

SEQ ID NO: 74 - protein, 14 aa, Artificial Sequence
  synthetic
  misc_feature (2)..(2): Xaa can be any naturally occurring amino acid
  misc_feature (6)..(6): Xaa can be any naturally occurring amino acid
  misc_feature (11)..(11): Xaa can be any naturally occurring amino acid
SXGAEXRTESXIHR

SEQ ID NO: 75 - protein, 14 aa, Artificial Sequence
  synthetic
  misc_feature (6)..(6): Xaa can be any naturally occurring amino acid
  misc_feature (11)..(11): Xaa can be any naturally occurring amino acid
SAGAEXRTESXIHR